(19) United States
(12) Patent Application Publication
(10) Pub. No.: US 2016/0000843 A1
(43) Pub. Date: Jan. 7, 2016

(54) HIGH CANNABIDIOL CANNABIS STRAINS

(71) Applicant: MJAR Holdings, LLC, Miami, FL (US)

(72) Inventors: James Lowe, Denver, CO (US); Matthew Curran, Denver (CO); Benjamin Franz, Denver (CO)

(73) Assignee: MJAR Holdings, LLC, Miami, FL (US)

(21) Appl. No.: 14/755,275

(22) Filed: Jun. 30, 2015

Related U.S. Application Data
(60) Provisional application No. 62/019,810, filed on Jul. 1, 2014.
(60) Provisional application No. 62/031,538, filed on Jul. 31, 2014.

(51) Int. Cl. A61K 36/185; A61K 31/192; A61K 31/047

(52) U.S. Cl. CPC A61K 36/185; A61K 31/047; A61K 31/192

(57) ABSTRACT

The invention described herein relates to a cannabis cultivar that produces high concentrations of cannabidiol. The invention further relates to preparations and products derived from the cannabis cultivar. Also provided are methods of treating conditions that are treatable by cannabidiol. by administering a preparation or product derived from the cannabis cultivar.

### CROSS-REFERENCE TO OTHER APPLICATIONS

This application claims priority to U.S. Provisional Application Ser. No. 62/019,810, filed Jul. 1, 2014, and 62/031,538, filed Jul. 31, 2014, each of which is incorporated herein by reference in its entirety.

### BACKGROUND OF THE INVENTION

1. Field of the Invention

This invention is directed in part to cannabis cultivars with high concentrations of cannabidiol and/or cannabidiolic acid and low concentrations of Δ9-tetrahydrocannabinol and/or tetrahydrocannabinolic acid.

2. State of the Art

Cannabis has long been considered to have medicinal properties. Many states, such as Colorado, Washington, Oregon, California, Alaska, Maine, Hawaii, Nevada, Vermont, Montana, Rhode Island, New Mexico, Michigan, New Jersey, allow the use of medicinal cannabis by persons with debilitating medical conditions as certified by physicians.

Cannabinoids, which are compounds derived from cannabis, are a group of chemicals from Cannabis species, including Cannabis sativa, Cannabis ruderalis, and Cannabis indica plant that are known to activate cannabinoid receptors (i.e., CB1 and CB2) in cells. There are at least 85 different cannabinoids that can be isolated from cannabis. Plant cannabinoids are termed “phytocannabinoids.” Cannabinoids are also produced endogenously in humans and other animals and are termed “endocannabinoids.” “Synthetic cannabinoids” are manmade chemicals with the same/similar structures as phytocannabinoids or endocannabinoids.

Cannabinoids are cyclic molecules exhibiting particular properties, such as the ability to easily cross the blood-brain barrier, weak toxicity and few side effects. The most notable cannabinoids produced by cannabis are Δ9-tetrahydrocannabinol (i.e., THC) and cannabidiol (i.e., CBD).

Some of the medical benefits attributable to one or more of the cannabinoids isolated from cannabis include treatment of pain, nausea, AIDS-related weight loss and wasting, multiple sclerosis, allergies, infection, depression, migraine, bipolar disorders, hypertension, post-stroke neuroprotection, epilepsy, and fibromyalgia, as well as inhibition of tumor growth, angiogenesis and metastasis. Studies have shown that cannabinoids may also be useful for treating conditions such as glaucoma, Parkinson's disease, Huntington's disease, migraines, inflammation, Crohn's disease, dystonia, rheumatoid arthritis, emesis due to chemotherapy, inflammatory bowel disease, atherosclerosis, posttraumatic stress disorder, cardiac reperfusion injury, prostate carcinoma, and Alzheimer's disease. For example, U.S. Pat. No. 6,630,507 discloses cannabinoids for use as antioxidants and neuroprotectants; U.S. Pat. No. 7,105,685 discloses cannabinoids for the treatment of diseases associated with immune dysfunction, particularly HIV disease and neoplastic disorders; U.S. Pat. No. 7,109,245 discloses cannabinoids useful as vasoconstrictors; U.S. Pat. Publication US2011/0257256 discloses THC-CBD composition for use in treating or preventing Cognitive Impairment and Dementia; PCT Publication WO/2009/147439 discloses use of cannabinoids in the manufacture of a medicament for use in the treatment of cancer, in particular the glioma tumor; PCT Publication WO/2007/148094 discloses use of cannabinoids composition for the treatment of neuropathic pain; and U.S. Pat. Publication US2010/0286098 discloses a method of treating tissue injury in a patient with colitis administering the cannabinoids.

While a wide range of medical uses has been identified, the benefits achieved by cannabinoids for a particular disease or condition are believed to be attributable to a subgroup of cannabinoids or to individual cannabinoids. That is to say that different subgroups or single cannabinoids have beneficial effects on certain conditions, while other subgroups or individual cannabinoids have beneficial effects on other conditions. For example, THC is the main psychoactive cannabinoid produced by Cannabis and is well-characterized for its biological activity and potential therapeutic application in a broad spectrum of diseases. CBD, another major cannabinoid constituent of Cannabis, acts as an inverse agonist of the CB1 and CB2 cannabinoid receptors. Unlike THC, CBD does not produce psychoactive effects in humans. CBD is reported to exert analgesic, antioxidant, anti-inflammatory, and immunomodulatory effects.

Terpenes, including terpenoids, are another class of compounds that are produced by cannabis. Reportedly, as many as 200 or more terpenes can be produced by cannabis plants, although the types and ratios of terpenes produced by a cannabis strain are dependent on genetics and growth conditions (e.g., lighting, fertilization, soil, watering frequency/amount, humidity, carbon dioxide concentration, and the like), as well as age, maturation, and time of day. Terpenes have been shown to have medicinal properties, and may be responsible for at least a portion of the medicinal value of cannabis.

Some of the medical benefits attributable to one or more of the terpenes isolated from cannabis include treatment of sleep disorders, psychosis, anxiety, epilepsy and seizures, pain, microbial infections (fungal, bacterial, etc.), cancer, inflammation, spasms, gastric reflux, depression, and asthma. Some terpenes have been shown to: lower the resistance across the blood-brain barrier, act on cannabinoid receptors and other neuronal receptors, stimulate the immune system, and/or suppress appetite.

To date, however, medicinal marijuana is used as a generic product whereby the patient utilizes the entirety of the different cannabinoids to achieve medicinal results. Efforts have been made to maximize the medicinal benefit of cannabis for a patient having a particular condition, but such efforts are invariably complicated. For example, because THC is psychoactive, some patients and regulatory authorities view cannabis with high CBD (and low THC) as being an alternative to traditional marijuana that is acceptable, legally, medically, and/or culturally. Additionally, cannabis employed by a patient lacks consistent cannabinoid components and concentrations, and thereby fails to provide the maximum benefit to the patient.

Cannabis expresses a large number of cannabinoids which are useful in the treatment of a variety of diseases. However, the usefulness of a cannabis cultivar for a particular disease is dependent upon the concentration of one or more specific cannabinoids, and/or the ratio between amounts of cannabinoids, produced by the cultivar.

### SUMMARY OF THE INVENTION

Cannabis and products or preparations thereof can be used to treat a variety of medical conditions in patients. However, the effectiveness of a given cannabis strain or cultivar in the treatment of a certain medical condition or symptom is dependent on the type(s) of cannabinoids present in the cultivar, strain, or preparation, both with respect to the amount of given cannabinoids and the ratios thereof. Cannabinoid types and concentrations are dependent on a number of factors, including cultivar or strain type (e.g., genetic background), growth conditions, harvest conditions, and methods of preparation.

Cannabidiol (CBD) has been implicated in the treatment of a wide variety of diseases and symptoms, including cancer, nausea, chronic pain, spasms, seizures/epilepsy, anxiety, psoriasis, Crohn's disease, rheumatoid arthritis, diabetes, schizophrenia, post-traumatic stress disorder (PTSD), alcoholism, strokes, Multiple Sclerosis, and cardiovascular disease. CBD also has been reported to act as a muscle relaxant, antibiotic, anti-inflammatory, and bone stimulant, as well as to improve blood circulation, cause drowsiness, and protect the nervous system.

Δ9-Tetrahydrocannabinol (THC) is also implicated in the treatment of disease. However, the psychotropic activity of THC makes it undesirable for some patients and/or indications.

This invention relates to a cannabis cultivar with a high CBD concentration but low THC concentration. This achieves the desire of patients to be treated with CBD without the side-effects (e.g., psychoactivity) of THC.

This invention is further predicated on the discovery that uniform cultivation parameters between batches of the same cannabis cultivar allows for substantially the same cannabinoid composition to be obtained from the same cultivar, regardless of batch. Cultivation parameters include, for example and without limitation, light intensity, light duration, fertilizer timing, fertilizer composition, watering schedules, watering quantity, amount of media, container size used, propagation methods, harvesting protocols, carbon dioxide concentrations, etc. Other considerations include water quality, pruning, plant support (e.g., trellising), pesticides and pest management, repotting, drying/curing, product storage, and the like. Implementation of the standardized processes described herein leads to improvement of the quality, uniformity, yield predictability and potential of the cultivars. In some embodiments, the cannabinoid composition of the harvested cannabis is substantially the same in each batch.

In one aspect, this invention is directed to a cannabis cultivar that produces an assayable combined cannabidiolic acid and cannabidiol concentration of at least about 18% by weight. In one embodiment, the cannabis cultivar produces an assayable combined cannabidiolic acid and cannabidiol concentration of at least about 20% by weight. In one embodiment, the cannabis cultivar produces an assayable combined cannabidiolic acid and cannabidiol concentration of between about 18% to about 60% by weight. In one embodiment, the cannabis cultivar produces an assayable combined cannabidiolic acid and cannabidiol concentration of between about 20% to about 60% by weight.

In one embodiment, the cannabis cultivar produces an assayable combined Δ9-tetrahydrocannabinol and tetrahydrocannabinolic acid concentration of less than about 3% by weight. In one embodiment, the cannabis cultivar produces an assayable combined Δ9-tetrahydrocannabinol and tetrahydrocannabinolic acid concentration of less than about 2% by weight. In one embodiment, the cannabis cultivar produces an assayable combined Δ9-tetrahydrocannabinol and tetrahydrocannabinolic acid concentration of less than about 1% by weight. In a preferred embodiment, the cannabis cultivar produces an assayable combined Δ9-tetrahydrocannabinol and tetrahydrocannabinolic acid concentration of less than about 0.1% by weight.

In one embodiment, the cannabis cultivar produces an estimated active cannabidiol concentration is at least about 18% by weight. In one embodiment, the cannabis cultivar produces an estimated active cannabidiol concentration is at least about 20% by weight.

In one embodiment, the cannabis cultivar produces an active Δ9-tetrahydrocannabinol concentration of less than about 3% by weight. In one embodiment, the cannabis cultivar produces an active Δ9-tetrahydrocannabinol concentration of less than about 2% by weight. In one embodiment, the cannabis cultivar produces an active Δ9-tetrahydrocannabinol concentration of less than about 1% by weight. In a preferred embodiment, the cannabis cultivar produces an active Δ9-tetrahydrocannabinol concentration of less than about 0.1% by weight.

In one aspect, this invention is directed to a preparation of the cannabis cultivar, wherein the preparation is a flower, an extract, an oil, an edible, a kief, an infusion, a tincture, or a hashish.

In one aspect, this invention is directed to a method of treating a cannabidiol-treatable condition and/or symptom thereof in a patient in need thereof by administering a high-cannabidiol cannabis cultivar or preparation or product thereof to the patient.

### DETAILED DESCRIPTION OF THE PREFERRED EMBODIMENTS

It is to be understood that this invention is not limited to particular embodiments described, as such may, of course, vary. It is also to be understood that the terminology used herein is for the purpose of describing particular embodiments only, and is not intended to be limiting, since the scope of this invention will be limited only by the appended claims.

The detailed description of the invention is divided into various sections only for the reader's convenience and disclosure found in any section may be combined with that in another section. Unless defined otherwise, all technical and scientific terms used herein have the same meaning as commonly understood by one of ordinary skill in the art to which this invention belongs.

### Definitions

It must be noted that as used herein and in the appended claims, the singular forms “a”, “an”, and “the” include plural referents unless the context clearly dictates otherwise. Thus, for example, reference to “a compound” includes a plurality of compounds.

Unless defined otherwise, all technical and scientific terms used herein have the same meaning as commonly understood by one of ordinary skill in the art to which this invention belongs. As used herein the following terms have the following meanings

The term “about” when used before a numerical designation, e.g., temperature, time, amount, concentration, and such other, including a range, indicates approximations which may vary by (+) or (−) 10%, 5% or 1%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

“Administration” refers to introducing an agent into a patient. Typically, an effective amount is administered, which amount can be determined by the treating physician or the like. Any route of administration, such as oral, topical, inhalation, nasal, buccal, sublingual, intranasal, or intrapulmonary can be used.

The related terms and phrases “administering” and “administration of”, when used in connection with a compound or pharmaceutical composition (and grammatical equivalents) refer both to direct administration, which may be administration to a patient by a medical professional or by self-administration by the patient, and/or to indirect administration, which may be the act of prescribing a drug. For example, a physician who instructs a patient to self-administer a drug and/or provides a patient with a prescription for a drug is administering the drug to the patient.

“Atomizing,” as used herein, means forming a spray. The term refers to the act of converting a liquid into fine particles suspended within the air. Atomizing is a general term which encompasses both vaporizing and aerosolizing. In at least some embodiments provided herein, atomizing involves converting a liquid oil into a suspension or dispersion of fluid particles each having a diameter of no more than 10 gm.

“Cannabis,” “cannabis species,” or “marijuana” refers to a flowering plant including the species (or sub-species) Cannabis sativa, Cannabis ruderalis, and Cannabis indica.

“Cannabinoids” refers to a class of chemical compounds that act on the cannabinoid receptors. “Endocannabinoids” are produced naturally in animals, including humans. “Phytocannabinoids” are naturally-occurring cannabinoids produced in plants. “Synthetic cannabinoids” are artificially manufactured cannabinoids.

Cannabis species express at least 85 different phytocannabinoids, which are concentrated in resin produced in glandular trichomes. The phytocannabinoids are divided into subclasses based on structure, including cannabigerols, cannabichromenes, cannabidiols, tetrahydrocannabinols, cannabinols and cannabinodiols, and other cannabinoids.

Cannabinoids found in cannabis include, without limitation: cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), and tetrahydrocannabivarinic acid (THCVA). Phytocannabinoids and their structures are discussed in more detail in U.S. Patent Application Pub. No. 2013/0059018, which is incorporated herein by reference in its entirety.

Phytocannabinoids can occur as either the pentyl(5 carbon atoms) or propyl(3 carbon atoms) variant. The propyl and pentyl variants may have distinct properties from one another. For example, THC is a CB1 receptor agonist, whereas the propyl variant THCV is a CB1 receptor antagonist meaning that it has almost opposite effects from THC.

“Terpenes” or “terpenoids” refers to a class of chemicals produced by plants, including cannabis. The term “terpenoid” generally refers to a chemically modified terpene (e.g., by oxidation). As used herein, the terpenes include terpenoids. Terpenes and terpenoids are often aromatic hydrocarbons and may have strong smells associated with them.

Terpenes known to be produced by cannabis include, without limitation, aromadendrene, bergamottin, bergamotol, bisabolene, borneol, 4-3-carene, caryophyllene, cineole/eucalyptol, p-cymene, dihydroj asmone, elemene, farnesene, fenchol, geranylacetate, guaiol, humulene, isopulegol, limonene, linalool, menthone, menthol, menthofuran, myrcene, nerylacetate, neomenthylacetate, ocimene, perillylalcohol, phellandrene, pinene, pulegone, sabinene, terpinene, terpineol, terpineol-4-ol, terpinolene, and derivatives, isomers, enantiomers, etc. of each thereof.

Cannabis plants and products may also comprise other pharmaceutically relevant compounds, including flavonoids and phytosterols (e.g., apigenin, quercetin, cannflavin A, β-sitosterol and the like).

“Products of cannabis” as used herein refers to any products derived from the cannabis plant, including but not limited to the flower, resin (hashish), and oil (hash oil), as well as any preparations thereof. Preparations include, by way of non-limiting example, dried flower, kief, hashish, tincture, hash oil, infusions, pipe resins, edibles, and the like.

As used herein, the term “flower,” “bud,” or “dried flower” refers to dried cannabis flowers, as well as the leaves (e.g., bracts) and stems associated therewith. This is the most widely consumed form of cannabis, and is often referred to as marijuana.

The term “kief” refers to a trichome-rich powder. It can be sifted from cannabis leaves and flowers. Trichomes are structures present on cannabis leaves, stems, and flowers that produce cannabinoids.

The term “hashish” or “hash” refers to a concentrated cake made from pressed kief.

The term “tincture” refers to cannabis extracts made using high-proof alcohol.

The term “hash oil” refers to oil extracted from cannabis flower and leaves.

The term “infusion” refers to infusion of cannabis in a variety of products. Non-limiting examples include tea, cocoa butter, dairy butter, cooking oil, glycerine, and other oils (e.g., skin moisturizers). Infusions include edibles like beer, soda, peanut butter, and the like.

The term “edible” refers to any cannabis product that can be consumed as food. In some cases, edibles are made by infusion of the cannabis into a foodstuff. In some cases, edibles are made by combining a cannabis product (e.g., dried flower, kief, hashish, tincture, hash oil, or infusion) with other ingredients to make an edible (e.g., a cookie, chocolate, lollipop, beer, popcorn, etc.).

“Yield potential” as used herein refers to the grams of product per square foot of cultivation space expected to be generated by a given cannabis strain or cultivar over a period of time. In a preferred embodiment, the period of time is the time from propagation to harvest of a cannabis plant or batch.

Cannabis plants go through a vegetative stage of growth, followed by a flowering cycle. The period of growth between germination or cutting rooting and flowering is known as the vegetative phase of plant development. Vegetation is the sporophytic state of the Cannabis plant. Plants do not produce flowers during the vegetative stage and are bulking up to a desired production size for flowering. During the vegetative phase, plants are busy carrying out photosynthesis and accumulating resources that will be needed for flowering and reproduction.

“Flowering cycle” or “flowering stage” (also called “bud cycle”) refers to the period during which the plant produces buds and flowers. This is the reproductive phase of plant growth, cannabis is dioecious having female and male reproduction parts on separate plants. Flowering is the gametophytic or reproductive state of Cannabis. For production, only females are selected for cultivation. For some cultivars, the switch from the vegetative stage to the flowering stage is light-dependent. Some cultivars are autoflowering, meaning they switch to the flowering stage automatically (e.g., with age).

“Cannabis cultivar” refers to cannabis plants that have been selected for one or more desirable characteristics and propagated. Where the term cultivar is used, it is to be understood that the cultivar may be a result of breeding and/or the result of genetic manipulation. A cannabis cultivar as described herein is not naturally-occurring. Propagation may occur in any manner, including, without limitation, sexual reproduction (e.g., seed), cloning (e.g., cuttings, vegetative propagation), self-pollinization, and the like.

A “plurality” as used herein refers to more than one. For example, a plurality of cannabinoids may be two, three, four, five, or more cannabinoids.

The term “active cannabinoid” as used herein refers to the non-acid form of the cannabinoid plus the amount of non-acid form estimated to be formed upon decarboxylation of the acid form.

Cannabinoids in their acid forms (e.g., CBDA or THCA) can be converted to their non-acid forms (e.g., THC or CBD) by decarboxylation. Decarboxylation occurs when the cannabinoid is heated. In addition, cannabinoid acid forms have been shown to have therapeutic activity. Cannabinoids lose mass when they are converted from the acid to non-acid (“active”) form. In order to determine the estimated amount of active cannabinoid that will be present after decarboxylation, the amount of the acid form can be multiplied by 87.7%. This is a rough estimate, and the actual amount of active cannabinoid that will be produced may be dependent upon the cannabinoid, method of decarboxylation, further breakdown of the cannabinoid during the decarboxylation process (e.g., due to heat), etc.

In one embodiment, “therapeutically effective amount” refers to that amount of a compound that results in prevention or amelioration of symptoms in a patient or a desired biological outcome, e.g., improved clinical signs, delayed onset of disease, etc. The effective amount can be determined by one of ordinary skill in the art. The selected dosage level can depend upon factors including, but not limited to, the severity of the condition being treated, and the condition and prior medical history of the patient being treated. However, it is within the skill of the art to start doses of the compound at levels lower than required to achieve the desired therapeutic effect and to gradually increase the dosage until the desired effect is achieved.

The term “modulate” or “modulating” means any treatment of a disease or disorder in a subject, such as a mammal, including:
preventing or protecting against the disease or disorder, that is, causing the abnormal biological reaction or symptoms not to develop;
inhibiting the disease or disorder, that is, arresting or suppressing the development of abnormal biological reactions and/or clinical symptoms; and/or
relieving the disease or disorder that is, causing the regression of abnormal biological reactions and/or clinical symptoms.

As used herein, the term “preventing” refers to the prophylactic treatment of a patient in need thereof. The prophylactic treatment can be accomplished by providing an appropriate dose of a therapeutic agent to a subject at risk of suffering from an ailment, thereby substantially averting onset of the ailment.

As used herein, the term “condition” refers to a disease state for which the compounds, compositions and methods provided herein are being used.

As used herein, the term “patient” or “subject” refers to mammals and includes humans and non-human mammals. In particular embodiments herein, the patient or subject is a human.

### High Cannabidiol Cannabis

In one aspect, described herein is a cannabis cultivar that produces high levels of CBD (and/or CBDA) and low levels of THC (and/or THCA). In one embodiment, the cannabis cultivar produces an assayable combined cannabidiolic acid and cannabidiol concentration of about 18% to about 60% by weight. In one embodiment, the cannabis cultivar produces an assayable combined cannabidiolic acid and cannabidiol concentration of about 20% to about 40% by weight. In one embodiment, the cannabis cultivar produces an assayable combined cannabidiolic acid and cannabidiol concentration of about 20% to about 30% by weight. In one embodiment, the cannabis cultivar produces an assayable combined cannabidiolic acid and cannabidiol concentration of about 25% to about 35% by weight. It should be understood that any subvalue or subrange from within the values described above are contemplated for use with the embodiments described herein.

In one embodiment, the cannabis cultivar produces an assayable combined cannabidiolic acid and cannabidiol concentration of at least about 18% by weight. In one embodiment, the cannabis cultivar produces an assayable combined cannabidiolic acid and cannabidiol concentration of at least about 19% by weight. In a preferred embodiment, the cannabis cultivar produces an assayable combined cannabidiolic acid and cannabidiol concentration of at least about 20% by weight. In one embodiment, the cannabis cultivar produces an assayable combined cannabidiolic acid and cannabidiol concentration of at least about 21% by weight. In one embodiment, the cannabis cultivar produces an assayable combined cannabidiolic acid and cannabidiol concentration of at least about 22% by weight. In one embodiment, the cannabis cultivar produces an assayable combined cannabidiolic acid and cannabidiol concentration of at least about 23% by weight. In one embodiment, the cannabis cultivar produces an assayable combined cannabidiolic acid and cannabidiol concentration of at least about 24% by weight. In one embodiment, the cannabis cultivar produces an assayable combined cannabidiolic acid and cannabidiol concentration of at least about 25% by weight. In one embodiment, the cannabis cultivar produces an assayable combined cannabidiolic acid and cannabidiol concentration of at least about 26% by weight. In one embodiment, the cannabis cultivar produces an assayable combined cannabidiolic acid and cannabidiol concentration of at least about 27% by weight. In one embodiment, the cannabis cultivar produces an assayable combined cannabidiolic acid and cannabidiol concentration of at least about 28% by weight. In one embodiment, the cannabis cultivar produces an assayable combined cannabidiolic acid and cannabidiol concentration of at least about 29% by weight. In one embodiment, the cannabis cultivar produces an assayable combined cannabidiolic acid and cannabidiol concentration of at least about 30% by weight.

In one embodiment, the cannabis cultivar produces an assayable combined Δ9-tetrahydrocannabinol and tetrahydrocannabinolic acid concentration of between about 0% to about 3% by weight. In one embodiment, the cannabis cultivar produces an assayable combined Δ9-tetrahydrocannabinol and tetrahydrocannabinolic acid concentration of between about 0% to about 2% by weight. In one embodiment, the cannabis cultivar produces an assayable combined Δ9-tetrahydrocannabinol and tetrahydrocannabinolic acid concentration of between about 0% to about 1% by weight. In one embodiment, the cannabis cultivar produces an assayable combined Δ9-tetrahydrocannabinol and tetrahydrocannabinolic acid concentration of between about 0% to about 0.3% by weight. In one embodiment, the cannabis cultivar produces an assayable combined Δ9-tetrahydrocannabinol and tetrahydrocannabinolic acid concentration of between about 0% to about 0.1% by weight. In one embodiment, the cannabis cultivar produces an assayable combined Δ9-tetrahydrocannabinol and tetrahydrocannabinolic acid concentration of between about 0% to about 0.09% by weight. In one embodiment, the cannabis cultivar produces an assayable combined Δ9-tetrahydrocannabinol and tetrahydrocannabinolic acid concentration of between about 0% to about 0.08% by weight. In one embodiment, the cannabis cultivar produces an assayable combined Δ9-tetrahydrocannabinol and tetrahydrocannabinolic acid concentration of between about 0% to about 0.07% by weight. In one embodiment, the cannabis cultivar produces an assayable combined Δ9-tetrahydrocannabinol and tetrahydrocannabinolic acid concentration of between about 0% to about 0.06% by weight. In one embodiment, the cannabis cultivar produces an assayable combined Δ9-tetrahydrocannabinol and tetrahydrocannabinolic acid concentration of between about 0% to about 0.05% by weight. In one embodiment, the cannabis cultivar produces an assayable combined Δ9-tetrahydrocannabinol and tetrahydrocannabinolic acid concentration of between about 0% to about 0.04% by weight. In one embodiment, the cannabis cultivar produces an assayable combined Δ9-tetrahydrocannabinol and tetrahydrocannabinolic acid concentration of between about 0% to about 0.03% by weight. In one embodiment, the cannabis cultivar produces an assayable combined Δ9-tetrahydrocannabinol and tetrahydrocannabinolic acid concentration of between about 0% to about 0.02% by weight. In one embodiment, the cannabis cultivar produces an assayable combined Δ9-tetrahydrocannabinol and tetrahydrocannabinolic acid concentration of between about 0% to about 0.01% by weight. In one embodiment, the cannabis cultivar produces an assayable combined Δ9-tetrahydrocannabinol and tetrahydrocannabinolic acid concentration of between about 0.02% to about 3% by weight. In a preferred embodiment, the cannabis cultivar produces an assayable combined Δ9-tetrahydrocannabinol and tetrahydrocannabinolic acid concentration of between about 0.02% to about 2% by weight. In one embodiment, the cannabis cultivar produces an assayable combined Δ9-tetrahydrocannabinol and tetrahydrocannabinolic acid concentration of between about 0.03% to about 2% by weight. In one embodiment, the cannabis cultivar produces an assayable combined Δ9-tetrahydrocannabinol and tetrahydrocannabinolic acid concentration of between about 0.05% to about 2% by weight. In one embodiment, the cannabis cultivar produces an assayable combined Δ9-tetrahydrocannabinol and tetrahydrocannabinolic acid concentration of between about 0.07% to about 2% by weight. It should be understood that any subvalue or subrange from within the values described above are contemplated for use with the embodiments described herein.

In one embodiment, the cannabis cultivar produces an assayable combined Δ9-tetrahydrocannabinol and tetrahydrocannabinolic acid concentration of less than about 3% by weight. In one embodiment, the cannabis cultivar produces an assayable combined Δ9-tetrahydrocannabinol and tetrahydrocannabinolic acid concentration of less than about 2% by weight. In one embodiment, the cannabis cultivar produces an assayable combined Δ9-tetrahydrocannabinol and tetrahydrocannabinolic acid concentration of less than about 1.9% by weight. In one embodiment, the cannabis cultivar produces an assayable combined Δ9-tetrahydrocannabinol and tetrahydrocannabinolic acid concentration of less than about 1.8% by weight. In one embodiment, the cannabis cultivar produces an assayable combined Δ9-tetrahydrocannabinol and tetrahydrocannabinolic acid concentration of less than about 1.7% by weight. In one embodiment, the cannabis cultivar produces an assayable combined Δ9-tetrahydrocannabinol and tetrahydrocannabinolic acid concentration of less than about 1.6% by weight. In one embodiment, the cannabis cultivar produces an assayable combined Δ9-tetrahydrocannabinol and tetrahydrocannabinolic acid concentration of less than about 1.5% by weight. In one embodiment, the cannabis cultivar produces an assayable combined Δ9-tetrahydrocannabinol and tetrahydrocannabinolic acid concentration of less than about 1.4% by weight. In one embodiment, the cannabis cultivar produces an assayable combined Δ9-tetrahydrocannabinol and tetrahydrocannabinolic acid concentration of less than about 1.3% by weight. In one embodiment, the cannabis cultivar produces an assayable combined Δ9-tetrahydrocannabinol and tetrahydrocannabinolic acid concentration of less than about 1.2% by weight. In one embodiment, the cannabis cultivar produces an assayable combined Δ9-tetrahydrocannabinol and tetrahydrocannabinolic acid concentration of less than about 1% by weight. In one embodiment, the cannabis cultivar produces an assayable combined Δ9-tetrahydrocannabinol and tetrahydrocannabinolic acid concentration of less than about 0.5% by weight. In one embodiment, the cannabis cultivar produces an assayable combined Δ9-tetrahydrocannabinol and tetrahydrocannabinolic acid concentration of less than about 0.3% by weight. In one embodiment, the cannabis cultivar produces an assayable combined Δ9-tetrahydrocannabinol and tetrahydrocannabinolic acid concentration of less than about 0.2% by weight. In one embodiment, the cannabis cultivar produces an assayable combined Δ9-tetraydrocanabinol and tetrahydrocannabinolic acid concentration of less than about 0.1% by weight. . In one embodiment, the cannabis cultivar produces an assayable combined Δ9-tetrahydrocannabinol and tetrahydrocannabinolic acid concentration of less than about 0.09% by weight. . In one embodiment, the cannabis cultivar produces an assayable combined Δ9-tetrahydrocannabinol and tetrahydrocannabinolic acid concentration of less than about 0.08% by weight. . In one embodiment, the cannabis cultivar produces an assayable combined Δ9-tetrahydrocannabinol and tetrahydrocannabinolic acid concentration of less than about 0.07% by weight. . In one embodiment, the cannabis cultivar produces an assayable combined Δ9-tetrahydrocannabinol and tetrahydrocannabinolic acid concentration of less than about 0.06% by weight. In one embodiment, the cannabis cultivar produces an assayable combined Δ9-tetrahydrocannabinol and tetrahydrocannabinolic acid concentration of less than about 0.05% by weight. . In one embodiment, the cannabis cultivar produces an assayable combined Δ9-tetrahydrocannabinol and tetrahydrocannabinolic acid concentration of less than about 0.04% by weight. . In one embodiment, the cannabis cultivar produces an assayable combined Δ9-tetrahydrocannabinol and tetrahydrocannabinolic acid concentration of less than about 0.03% by weight. . In one embodiment, the cannabis cultivar produces an assayable combined Δ9-tetrahydrocannabinol and tetrahydrocannabinolic acid concentration of less than about 0.02% by weight. . In one embodiment, the cannabis cultivar produces an assayable combined Δ9-tetrahydrocannabinol and tetrahydrocannabinolic acid concentration of less than about 0.01% by weight. In one embodiment, the cannabis cultivar produces an assayable combined Δ9-tetrahydrocannabinol and tetrahydrocannabinolic acid concentration of about 0.07% by weight.

In one embodiment, the estimated active cannabidiol concentration is between about 18% and about 60% by weight. In one embodiment, the estimated active cannabidiol concentration is between about 20% and about 60% by weight. In one embodiment, the estimated active cannabidiol concentration is between about 20% and about 50% by weight. In one embodiment, the estimated active cannabidiol concentration is between about 20% and about 40% by weight. In one embodiment, the estimated active cannabidiol concentration is between about 20% and about 30% by weight. In one embodiment, the estimated active cannabidiol concentration is between about 20% and about 25% by weight. In one embodiment, the estimated active cannabidiol concentration is between about 25% and about 40% by weight. In one embodiment, the estimated active cannabidiol concentration is between about 25% and about 30% by weight. It should be understood that any subvalue or subrange from within the values described above are contemplated for use with the embodiments described herein.

In one embodiment, the estimated active cannabidiol concentration is at least about 18% by weight. In one embodiment, the estimated active cannabidiol concentration is at least about 19% by weight. In one embodiment, the estimated active cannabidiol concentration is at least about 20% by weight. In one embodiment, the estimated active cannabidiol concentration is at least about 21% by weight. In one embodiment, the estimated active cannabidiol concentration is at least about 22% by weight. In one embodiment, the estimated active cannabidiol concentration is at least about 23% by weight. In one embodiment, the estimated active cannabidiol concentration is at least about 24% by weight. In one embodiment, the estimated active cannabidiol concentration is at least about 25% by weight. In one embodiment, the estimated active cannabidiol concentration is at least about 26% by weight. In one embodiment, the estimated active cannabidiol concentration is at least about 27% by weight. In one embodiment, the estimated active cannabidiol concentration is at least about 28% by weight. In one embodiment, the estimated active cannabidiol concentration is at least about 29% by weight. In one embodiment, the estimated active cannabidiol concentration is at least about 30% by weight.

In one aspect, this invention is directed to a cannabis strain or product thereof, wherein the ratio of estimated active cannabidiol to estimated active THC is between about 400:1 and about 15:1. In one embodiment, the ratio of active cannabidiol to active THC is between about 300:1 and about 20:1. In one embodiment, the ratio of active cannabidiol to active THC is between about 300:1 and about 25:1. In one embodiment, the ratio of active cannabidiol to active THC is between about 300:1 and about 30:1. In one embodiment, the ratio of active cannabidiol to active THC is between about 300:1 and about 50:1. In one embodiment, the ratio of active cannabidiol to active THC is between about 300:1 and about 70:1. In one embodiment, the ratio of active cannabidiol to active THC is between about 300:1 and about 80:1. In one embodiment, the ratio of active cannabidiol to active THC is between about 300:1 and about 100:1. In one embodiment, the ratio of active cannabidiol to active THC is between about 300:1 and about 200:1.

The concentration of cannabinoids can be determined based on a sample taken from any portion of the cannabis plant. The concentration of cannabinoids can be determined based on a sample taken at any point in the life cycle of the plant. In a preferred embodiment, the sample is taken from a flower (or inflorescence) of a cannabis plant. In one embodiment, the sample is taken from one or more flowers, leaves, stems, or a combination thereof. In one embodiment, the sample is taken during a vegetative stage of the cannabis life cycle. In one embodiment, the sample is taken during the flowering stage of the cannabis life cycle.

In one aspect of the invention, the cannabis cultivar is cultivated by a method as described in U.S. patent application Ser. No. 14/745,358, which is incorporated herein by reference in its entirety.

### Products and Preparations

The cannabis cultivar provided herein can be processed into a variety of products or preparations. Generally, at least a portion of a cannabis plant is processed, for example a flower, inflorescence, leaf, and/or stem. In one embodiment, the portion of the plant is harvested and dried prior to further processing.

Oil, cannabinoids, and/or other compounds can be extracted from portions of the cannabis plant using known extraction methods. By way of non-limiting example, a portion of the cannabis plant can be contacted with one or more solvents (including, but not limited to, butane, propane, carbon dioxide, and/or alcohol, e.g., ethanol). In a preferred embodiment, the solvent is an alcohol. In an especially preferred embodiment, the alcohol is ethanol.

Portions of the cannabis plant that can be used to extract cannabinoids include the flowers, inflorescences, leaves, and/or stems. The plant or portion thereof to be extracted is optionally dried prior to extraction. In one embodiment, a portion of the cannabis plant is mechanically processed (e.g., cut, sifted, or ground). Where the portion of the cannabis plant is mechanically processed, the resulting material may be used directly (e.g., to administer to a patient, as an ingredient in an edible, etc.) or may be extracted or otherwise further processed prior to use.

After the desired portion(s) of the cannabis plant are processed, the resulting material may be used to make cannabis preparations. In one aspect, this invention relates to a preparation of a cannabis plant as described herein. In one embodiment, the preparation is a flower or a dried flower. In one embodiment, the preparation is an extract. In one embodiment, the preparation is a kief. In one embodiment, the preparation is an infusion. In one embodiment, the preparation is a tincture. In one embodiment, the preparation is a hashish. In one embodiment, the preparation is a topical formulation. In one embodiment, the preparation is a spray. In one embodiment, the preparation is a salve.

In a preferred embodiment, the preparation is an oil. In one embodiment, the oil comprises less than about 3% by weight of the solvent used for extraction of the cannabinoids. In one embodiment, the oil comprises less than about 2% by weight of the solvent used for extraction of the cannabinoids. In one embodiment, the oil comprises less than about 1% by weight of the solvent used for extraction of the cannabinoids. In one embodiment, the oil comprises less than about 0.5% by weight of the solvent used for extraction of the cannabinoids. In one embodiment, the oil comprises less than about 0.1% by weight of the solvent used for extraction of the cannabinoids. In one embodiment, the oil comprises less than about 0.01% by weight of the solvent used for extraction of the cannabinoids. In one embodiment, the oil comprises less than about 0.001% by weight of the solvent used for extraction of the cannabinoids.

In one embodiment, the preparation is an edible. The edible can be any foodstuff comprising cannabinoids derived from a cannabis plant as described herein. In one embodiment, the edible is a chocolate, popcorn, butter, cooking oil, cookie, pastry, bread, beer, tea, soda, mint, candy, lollipop, peanut butter, brownie, shake, concentrate, punch, cocoa, gummy candy, protein bar, candy bar, etc.

### Cannabinoid Composition

In one aspect, the cannabinoid and/or terpene composition of a strain of cannabis is consistent between batches. In one embodiment, the cannabinoid and/or terpene composition of a particular strain is consistent between batches grown and harvested at different times. In one embodiment, the cannabinoid and/or terpene composition of a particular strain is consistent between batches grown and/or harvested at different locations (e.g., different cultivation facilities). The term “consistent” means that the concentration of a given cannabinoid and/or terpene present in a particular strain does not vary by more than 20%, preferably 15%, 10%, or 5%. In one embodiment, the cannabinoid is a phytocannabinoid. In one embodiment, the cannabinoid is CBD. In one embodiment, the cannabinoid is THC. In one embodiment, the cannabinoid is CBN. In one embodiment, the cannabinoid is at least one of cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), or tetrahydrocannabivarinic acid (THCVA).

In one aspect, the cannabinoid and/or terpene composition of the strain is determined by assaying the concentration of at least one cannabinoid in a subset (e.g., sample) of the harvested product. In one embodiment, THC concentration is assayed. In one embodiment, CBD concentration is assayed. In one embodiment, the cannabinoid is CBN. In one embodiment, the cannabinoid is at least one of cannabigerol (CBG), cannabichromene (CBC), cannabidiol (CBD), tetrahydrocannabinol (THC), cannabinol (CBN) and cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), or tetrahydrocannabivarinic acid (THCVA). In one embodiment, the concentration of one or more terpenes is assayed.

Assays used heretofore to determine cannabinoid concentrations have shown significant deviation for the same plant. Such deviation is a problem for the cannabis industry as a whole, and particularly the medical marijuana industry, which requires reproducibility. After careful examination, it is contemplated that the moisture content of the assayed composition is a critical parameter in the variability of the assayed concentrations. Surprisingly, such variability can be minimized by rendering the moisture content consistent from assay to assay. In one aspect, this invention relates to a method of assaying cannabinoid concentration of a cannabis sample such that the assay provides reproducible results between different samples, e.g., batches and/or strains.

In one embodiment, the moisture content of a sample to be tested (e.g., for cannabinoid content) is adjusted to a consistent level prior to performing the assay. In one embodiment, a sample to be tested is adjusted to 40% moisture or less. In one embodiment, a sample to be tested is adjusted to about 40% moisture. In one embodiment, a sample to be tested is adjusted to about 30% moisture. In one embodiment, a sample to be tested is adjusted to about 20% moisture. In one embodiment, a sample to be tested is adjusted to about 15% moisture. In one embodiment, a sample to be tested is adjusted to about 14% moisture. In one embodiment, a sample to be tested is adjusted to about 13% moisture. In one embodiment, a sample to be tested is adjusted to about 12% moisture. In one embodiment, a sample to be tested is adjusted to about 11% moisture. In one embodiment, a sample to be tested is adjusted to about 10% moisture. In one embodiment, a sample to be tested is adjusted to about 9% moisture. In one embodiment, a sample to be tested is adjusted to about 8% moisture. In one embodiment, a sample to be tested is adjusted to about 7% moisture. In one embodiment, a sample to be tested is adjusted to about 6% moisture. In one embodiment, a sample to be tested is adjusted to about 5% moisture. In yet another approach, the composition is lyophilized prior to assay. Methods for determining moisture content are well-known in the art.

The moisture content of a sample to be tested can be adjusted using any method for adjusting moisture content. In one embodiment, the moisture content is adjusted by placing the sample in a hydrator or humidity chamber at a desired humidity level for a period of time before it is assayed. In one embodiment, the moisture content is adjusted by dehydrating the sample to a desired moisture content. The sample can be dehydrated using any dehydration method. In one embodiment, the moisture content is adjusted by lyophilizing the sample. In one embodiment, the moisture content of more than one sample is adjusted at the same time. In one embodiment, the moisture content of the sample is determined before the adjusting step. In one embodiment, the moisture content of the sample is not determined before the adjusting step.

In one embodiment, the concentration of cannabinoids is determined irrespective of the moisture content. For example, the dry weight of the sample can be determined, and the cannabinoid content is determined relative to the dry weight.

After the desired moisture content has been achieved, the cannabinoid content can be determined using any method. Methods include, without limitation, radioimmunoassay, gas chromatography/mass spectrometry, gas chromatography, liquid chromatography, liquid chromatography/mass spectrometry, and enzyme immunoassay.

### Methods of Treatment

In one aspect, this invention relates to a method of treating a cannabidiol-treatable condition and/or symptom thereof in a patient in need thereof. In one embodiment, the method comprises administering to the patient an effective amount of the cannabis cultivar, cannabis product, or preparation as described above, wherein the condition and/or symptom is treated.

In one embodiment, the condition is a cancer, nausea, chronic pain, spasms, seizures, epilepsy, anxiety, psoriasis, Crohn's disease, rheumatoid arthritis, diabetes, schizophrenia, post-traumatic stress disorder, alcoholism, strokes, Multiple Sclerosis, or cardiovascular disease. In a preferred embodiment, spasms and/or seizures are treated. In another preferred embodiment, anxiety is treated. In one embodiment, a symptom of the condition is treated.

In one embodiment, the effective amount of the cannabis cultivar, cannabis product, or preparation acts as a muscle relaxant, antibiotic, anti-inflammatory, bone stimulant, improves blood circulation, causes drowsiness, and/or protects the nervous system.

In one embodiment, the administration of cannabis as described herein attenuates a symptom of a cannabidiol-treatable condition. In one embodiment, the administration of cannabis as described herein prevents a symptom of a cannabidiol-treatable condition. In one embodiment, the administration of cannabis as described herein modulates a symptom of a cannabidiol-treatable condition.

The compositions, provided herein or known, suitable for administration in accordance with the methods provided herein, can be suitable for a variety of delivery modes including, without limitation, transdermal, sublingual, intrapulmonary, or intranasal delivery. Compositions suitable for internal, pulmonary, and lingual routes may also be used. Other dosage forms include tablets, capsules, pills, powders, aerosols, suppositories, parenterals, and oral liquids, including suspensions, solutions and emulsions. Sustained release dosage forms may also be used. All dosage forms may be prepared using methods that are standard in the art (see e.g., Remington's Pharmaceutical Sciences, 16th ed., A. Oslo editor, Easton Pa. 1980).

Cannabis as described herein can also be used in conjunction with any of the vehicles and excipients commonly employed in pharmaceutical preparations, e.g., talc, gum Arabic, lactose, starch, magnesium stearate, cocoa butter, aqueous or non-aqueous solvents, oils, paraffin derivatives, glycols, etc. Coloring and flavoring agents may also be added to preparations, particularly to those for oral administration. Solutions can be prepared using water or physiologically compatible organic solvents such as ethanol, 1,2-propylene glycol, polyglycols, dimethylsulfoxide, fatty alcohols, triglycerides, partial esters of glycerine and the like. Parenteral compositions containing noribogaine may be prepared using conventional techniques that may include sterile isotonic saline, water, 1,3-butanediol, ethanol, 1,2-propylene glycol, polyglycols mixed with water, Ringer's solution, etc.

The compositions utilized herein may be formulated for aerosol administration, particularly to the respiratory tract and including intrapulmonary or intranasal administration. The compound will generally have a small particle size, for example of the order of 5 microns or less. Such a particle size may be obtained by means known in the art, for example by micronization. The active ingredient may be provided in a pressurized pack with a suitable propellant such as a chlorofluorocarbon (CFC), (for example, dichlorodifluoromethane, trichlorofluoromethane, or dichlorotetrafluoroethane), carbon dioxide or other suitable gases. The aerosol may conveniently also contain a surfactant such as lecithin. The dose of drug may be controlled by a metered valve. Alternatively, the active ingredients may be provided in the form of a dry powder, for example a powder mix of the compound in a suitable powder base such as lactose, starch, starch derivatives such as hydroxypropylmethyl cellulose and polyvinylpyrrolidine. In some embodiments, the powder carrier will form a gel in the nasal cavity. The powder composition may be presented in unit dose form, for example in capsules or cartridges, gelatin or blister packs, from which the powder may be administered by means of an inhaler.

In one aspect, the compositions utilized herein may be formulated for sublingual administration, for example as a tincture or sublingual tablets. Sublingual tablets are designed to dissolve very rapidly. The formulations of these tablets contain, in addition to the drug, a limited number of soluble excipients, usually lactose and powdered sucrose, but sometimes dextrose and mannitol.

In one aspect, cannabis as described herein may be inhaled. In one embodiment, cannabis as described herein may be administered in an atomized or nebulized form, for example by the use of an electronic cigarette, vaporizer, atomizer, or nebulizer. In one embodiment, the cannabis is smoked (i.e., burned and inhaled). In various embodiments, a drug delivery device converts a liquid comprising medicinal cannabis compounds, for example, an oil extract from cannabis strains as described herein, into a vapor or aerosol. In at least some embodiments, the inhalable drug delivery devices atomize the liquid while causing little or no combustion. Advantageously, inhalable cannabis compounds can be absorbed into the bloodstream almost immediately. The peak effect may be felt, for example, within 30 minutes or less from the time of inhalation. Such absorption times are a particular improvement over ingested cannabis delivery where the peak effect may not be felt for six or more hours.

In one embodiment, described herein is a composition comprising atomized cannabidiol and/or cannabidiolic acid. In one embodiment, the composition comprises one or more additional cannabinoids. In one embodiment, the composition comprises Δ9-tetrahydrocannabinol and/or tetrahydrocannabinolic acid. In one embodiment, the composition comprises at least one terpene. In one embodiment, the composition comprises one or more additional compounds made by cannabis.

In one embodiment, the composition comprises a ratio of cannabidiol and/or cannabidiolic acid (or active CBD) to Δ9-tetrahydrocannabinol and/or tetrahydrocannabinolic acid (or active THC) of about 25:1 to about 300:1. In one embodiment, the ratio of active cannabidiol to active THC is between about 300:1 and about 30:1. In one embodiment, the ratio of active cannabidiol to active THC is between about 300:1 and about 50:1. In one embodiment, the ratio of active cannabidiol to active THC is between about 300:1 and about 70:1. In one embodiment, the ratio of active cannabidiol to active THC is between about 300:1 and about 80:1. In one embodiment, the ratio of active cannabidiol to active THC is between about 300:1 and about 100:1. In one embodiment, the ratio of active cannabidiol to active THC is between about 300:1 and about 200:1.

In one aspect, the atomized composition is provided by any device capable of atomizing a cannabinoid-containing composition. In one embodiment, the atomized composition is provided by a nebulizer. In one embodiment, the atomized composition is provided by an electronic cigarette. In one embodiment, the atomized composition is provided by a vaporizer. In one embodiment, the atomized composition is provided by an atomizer. In one aspect, the atomized composition is administered to a patient. In one aspect, the atomized composition is suitable for pulmonary delivery to a patient.

In one aspect, cannabis is administered orally, for example incorporated into a food or beverage. In one embodiment, the edible is a chocolate, popcorn, butter, cooking oil, cookie, pastry, bread, beer, tea, soda, mint, candy, lollipop, peanut butter, brownie, shake, concentrate, punch, cocoa, gummy candy, protein bar, candy bar, etc.

In one aspect, cannabis is administered as a unit dose form. The term “unit dose” refers to a dose of cannabis that is given to the patient to provide therapeutic results, independent of the weight of the patient. In such an instance, the unit dose is sold in a standard form (e.g., 20 mg tablet). The unit dose may be administered as a single dose or a series of subdoses. In some embodiments, the unit dose provides a standardized level of drug to the patient, independent of weight of patient. In one embodiment, the unit dose is a single serving of a cannabis-infused food. In one embodiment, the unit dose is provided in transdermal form. In one embodiment, the unit dose is a tablet, caplet, or pill. In one embodiment, the unit dose is provided in an electronic cigarette cartridge. In one embodiment, the unit dose is provided by programming an electronic cigarette or vaporizer to administer the unit dose within a given period of time, number of uses, etc. In one embodiment, the unit dose is provided by a metered dose of a flower, an extract, an oil, an edible, a kief, an infusion, a tincture, or a hashish.

In one aspect, a unit dose comprises up to about 300 mg CBD and/or CBD-A. In one embodiment, a unit dose comprises up to about 250 mg CBD and/or CBD-A. In one embodiment, a unit dose comprises up to about 200 mg CBD and/or CBD-A. In one embodiment, a unit dose comprises up to about 150 mg CBD and/or CBD-A. In one embodiment, a unit dose comprises up to about 100 mg CBD and/or CBD-A. In one embodiment, a unit dose comprises up to about 50 mg CBD and/or CBD-A. In one embodiment, a unit dose comprises up to about 25 mg CBD and/or CBD-A. In one embodiment, a unit dose comprises up to about 20 mg CBD and/or CBD-A. In one embodiment, a unit dose comprises up to about 15 mg CBD and/or CBD-A. In one embodiment, a unit dose comprises up to about 10 mg CBD and/or CBD-A.

In one aspect, a unit dose comprises less than about 12 mg THC and/or THC-A. In one embodiment, a unit dose comprises less than about 10 mg THC and/or THC-A. In one embodiment, a unit dose comprises less than about 8 mg THC and/or THC-A. In one embodiment, a unit dose comprises less than about 6 mg THC and/or THC-A. In one embodiment, a unit dose comprises less than about 4 mg THC and/or THC-A. In one embodiment, a unit dose comprises less than about 2 mg THC and/or THC-A. In one embodiment, a unit dose comprises less than about 1 mg THC and/or THC-A. In one embodiment, a unit dose comprises less than about 0.8 mg THC and/or THC-A. In one embodiment, a unit dose comprises less than about 0.6 mg THC and/or THC-A. In one embodiment, a unit dose comprises less than about 0.4 mg THC and/or THC-A. In one embodiment, a unit dose comprises less than about 0.2 mg THC and/or THC-A. In one embodiment, a unit dose comprises less than about 0.1 mg THC and/or THC-A. In one embodiment, a unit dose comprises about 0 mg THC and/or THC-A.

### EXAMPLES

Additional embodiments are disclosed in further detail in the following examples, which are not in any way intended to limit the scope of the claims.

### Example 1

### Cannabis Cultivar A

The cannabis cultivar was harvested and dried, and an inflorescence was taken as a sample for analysis.

The sample was tested for cannabinoid content. The values are shown in Table 1. Total assayable cannabinoid concentration was determined to be approximately 22.79%. Moisture content was approximately 9.23%.

TABLE 1

| | |
|---|---|
| Assayable Cannabinoids | Active Cannabinoids |
| (% by weight) | (estimated; % by weight) |

| | | | |
|---|---|---|---|
| CBD-A | 21.77 | Max CBD | 20.03 |
| CBD | 0.94 | | |
| THC-A | 0.08 | Max THC | 0.07¹ |
| THC | <0.001 | | |
| CBN | <0.001 | | |

¹An initial reading provided a max THC value of 0.29%. However, the assay was repeated and the initial reading was found to be in error. The current reading is believed to be correct.

The sample contained less than 0.001% each of CBD-V, CBG, THC-V, and CBC.

1. A cannabis cultivar that produces an assayable combined cannabidiolic acid and cannabidiol concentration of at least about 20% by weight.

2. The cannabis cultivar of claim 1 that produces an assayable combined Δ9-tetrahydrocannabinol and tetrahydrocannabinolic acid concentration of less than about 0.1% by weight.

3. The cannabis cultivar of claim 1, wherein the estimated active cannabidiol concentration is at least about 20% by weight.

4. The cannabis cultivar of claim 1, wherein the active Δ9-tetrahydrocannabinol concentration is less than about 0.1% by weight.

5. The cannabis cultivar of claim 1, wherein the cannabinoid concentration is determined based on a moisture content of between about 5% and about 10%.

6. The cannabis cultivar of claim 5, wherein the cannabinoid concentration is determined based on a moisture content of between about 8% and about 10%.

7. A seed of the cannabis cultivar of claim 1.

8. A clone of the cannabis cultivar of claim 1.

9. A cannabis product that is a dehydrated portion of the cannabis cultivar of claim 1.

10. The cannabis product of claim 9, wherein the dehydrated portion is one or more of an inflorescence, a flower, a leaf, or a stem.

11. A preparation of the cannabis cultivar of claim 1, wherein the preparation is a flower, an extract, an oil, an edible, a kief, an infusion, a tincture, or a hashish.

12. The preparation of claim 11, wherein the preparation is an oil.

13. The preparation of claim 12, comprising less than about 1% by weight of a solvent used for extraction.

14. The preparation of claim 1 in a container that is impervious to visible light.

15. A method of treating a cannabidiol-treatable condition and/or symptom thereof in a patient in need thereof, the method comprising administering to the patient an effective amount of a composition comprising the cannabis cultivar of claim 1, wherein the condition and/or symptom is treated.

16. The method of claim 15, wherein the condition is selected from the group consisting of: cancer, nausea, chronic pain, spasms, seizures, epilepsy, anxiety, psoriasis, Crohn's disease, rheumatoid arthritis, diabetes, schizophrenia, post-traumatic stress disorder, alcoholism, strokes, Multiple Sclerosis, and cardiovascular disease.

17. A unit dose of the cannabis cultivar of claim 1.

18. The unit dose of claim 17 comprising up to about 300 mg of cannabidiol and/or cannabidiolic acid.

19. The unit dose of claim 17 comprising less than about 12 mg Δ9-tetrahydrocannabinol and/or tetrahydrocannabinolic acid.

20. A composition comprising atomized cannabidiol and/or cannabidiolic acid, wherein the ratio of atomized cannabidiol and/or cannabidiolic acid to atomized Δ9-tetrahydrocannabinol and/or tetrahydrocannabinolic acid is between about 300:1 and about 25:1.

21. The composition of claim 20, wherein the atomized cannabidiol and/or cannabidiolic acid is provided by an electronic cigarette, a nebulizer, or a vaporizer.
